(19) United States
(12) Patent Application Publication
(10) Pub. No.: US 2026/0341457 A1
(43) Pub. Date: Oct. 29, 2026

(54) MACHINE LEARNING TO PREDICT THERAPY PROGRESSION

(71) Applicant: RESMED DIGITAL HEALTH INC., San Diego, CA (US)

(72) Inventors: Zhuo Qi LEE, Singapore (SG); Yang YAN, Singapore (SG); Yuan YONGSHAN, Singapore (SG); Alena MELNIKOVA, Singapore (SG); Tan Yu JIA, Singapore (SG)

(21) Appl. No.: 19/118,214

(22) PCT Filed: Oct. 4, 2023
(86) PCT No.: PCT/US2023/076010

§ 371 Date: Apr. 3, 2025

Related U.S. Application Data
(60) Provisional application No. 63/413,142, filed on Oct. 4, 2022.

(51) Int. Cl. G16H 20/30; G06N 20/00

(52) U.S. Cl. CPC G16H 20/30; G06N 20/00

(57) ABSTRACT

Techniques for progress prediction using machine learning are provided. User data describing a user associated with a therapy plan is received, and a set of features is generated by applying one or more preprocessing transformations to the user data. A predicted therapy progress is generated for the user by processing the set of features using a trained machine learning model, where the predicted therapy progress comprises a stage, of a defined set of stages, associated with the therapy plan. Enrollment of the user in the therapy plan is facilitated based on the predicted therapy progress.

### CROSS-REFERENCE TO RELATED APPLICATIONS

This Application is a national phase application of PCT Application No. PCT/US 2023/076010, filed on Oct. 4, 2023, which claims the benefit of and priority to U.S. Provisional Patent Application No. 63/413,142, filed on Oct. 4, 2022, the entire contents of which are incorporated herein by reference.

### BACKGROUND

### Field

Embodiments of the present disclosure generally relate to machine learning. More specifically, embodiments of the present disclosure relate to training and using machine learning to predict therapy progression.

### Description of the Related Art

Defined sequences of actions or events are often used to track or manage progress in a wide variety of industries and practices. For example, in healthcare settings, there is often a sequence of events involved in enrolling or engaging in a therapy (e.g., to begin the therapy), as well as a sequence of events involved in actually participating in the therapy. In realistic environments, it is common for such schedules or plans to be begun, but not completed. For example, a user may begin the enrollment process to start therapy, and stop before actually beginning the therapy. Similarly, a user may begin therapy, but stop following the therapy plan before sufficient time has passed or before reaching their goals. Often, when such plans are terminated prematurely, the resources used are considered wasted. That is, any resources that were expended to reach the given stage of the plan before premature termination may have been better used to advance an alternative or other plan or sequence.

Accordingly, improved systems and techniques to monitor, manage, and/or predict therapy progression are needed.

### SUMMARY

According to one embodiment presented in this disclosure, a method is provided. The method includes: receiving user data describing a user associated with a therapy plan; determining an achieved stage, from a defined set of stages associated with the therapy plan, based on the user data; labeling the user data based on the achieved stage; training a machine learning model to predict therapy progress based on the user data and the achieved stage; and storing a data structure representing the machine learning model for runtime deployment.

According to one embodiment presented in this disclosure, a method is provided. The method includes: receiving user data describing a user associated with a therapy plan; generating a set of features by applying one or more preprocessing transformations to the user data; generating a predicted therapy progress for the user by processing the set of features using a trained machine learning model, wherein the predicted therapy progress comprises a stage, of a defined set of stages, associated with the therapy plan; and facilitating enrollment of the user in the therapy plan based on the predicted therapy progress.

Other aspects provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 depicts an example environment for using machine learning to predict and manage therapy progress.

FIG. 2 depicts an example workflow for training machine learning models to predict therapy progress.

FIG. 3 depicts an example workflow for using machine learning models to predict therapy progress.

FIG. 4 is a flow diagram depicting an example method for training machine learning models to predict therapy progress.

FIG. 5 is a flow diagram depicting an example method for using machine learning models to predict therapy progress.

FIG. 6 is a flow diagram depicting an example method for training machine learning models.

FIG. 7 is a flow diagram depicting an example method for predicting therapy progress.

FIG. 8 depicts an example computing device configured to perform various aspects of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for using machine learning to predict therapy progress.

In some embodiments, techniques and systems are described to enable training and use of machine learning models to predict patient or user compliance or progress with respect to positive airway pressure (PAP) therapies. For example, in one embodiment, the techniques described herein can be used to predict whether users will reach a given stage of enrolling in PAP therapy (e.g., whether they will actually complete enrollment and begin therapy).

In some embodiments, the PAP therapy enrollment process can involve stages such as asking the user to respond to a questionnaire or survey (e.g., discussing self-reported symptoms of apnea), receiving a response, identifying and engaging with users who may benefit from PAP therapy (based on the survey responses), selecting a device, provisioning the device, and/or actually beginning therapy. At each step of the therapy plan, however, there is potential for the user to cease participation. For example, the number of users that respond to the survey is generally significantly larger than the number who actually begin PAP therapy.

For each user who begins but does not complete the plan, resources are wasted. For example, computational resources are used to evaluate the user in order to determine their eligibility and/or whether they may benefit from therapy. Similarly, manual effort (e.g., from subject matter experts, such as sleep coaches) is expended attempting to connect with the users, identify or suggest appropriate therapy options, provision needed supplies, and the like. Moreover, physical healthcare resources are often used, such as to provide one or more PAP machines, to provide consumables or additional elements such as filters, tubes, masks, and the like.

In some embodiments of the present disclosure, machine learning models can be trained and/or used to predict user progress on a therapy plan. For example, a machine learning model may be trained to evaluate input such as the user's responses to survey questions, the demographics of the user (e.g., their age, race, ethnicity, gender, marital status, education, employment, and the like), and the like, in order to generate predicted progress measures (e.g., indicating the probability that they will reach a given stage of the plan, such as completing enrollment, indicating the stage they will likely reach in the plan, and the like).

By training and using such machine learning models, aspects of the present disclosure can reduce resource waste (which includes computational resources, manual resources, and physical resources). For example, by predicting which users are unlikely to complete enrollment, aspects of the present disclosure can prevent further resources from being used on such users. Similarly, by predicting which users are likely to complete enrollment and benefit from therapy, aspects of the present disclosure enable targeted resource usage and improved therapy outcomes for such patients.

In some aspects of the present disclosure, PAP therapy (e.g., using a continuous PAP (CPAP) device, a bi-level PAP (BiPAP) device, an automatic PAP (APAP) device, and the like) is used as an example plan (e.g., an example sequence of events or actions) that can be monitored, managed, and/or predicted using embodiments of the present disclosure. However, the presently-disclosed embodiments can generally be used to predict progression of any sequence of events or actions, such as progress towards enrolling in various systems (e.g., educational systems), progress in completing various tasks (e.g., in construction, in academic settings, in healthcare settings), and the like.

Additionally, though the probability of completing a plan or sequence of events is used as an example prediction in some embodiments, aspects of the present disclosure can generally be used to predict the progression of plans towards any goal (e.g., the probability that a user will reach or achieve any given stage in the plan).

### Example Environment for Using Machine Learning to Predict and Manage Therapy Progress

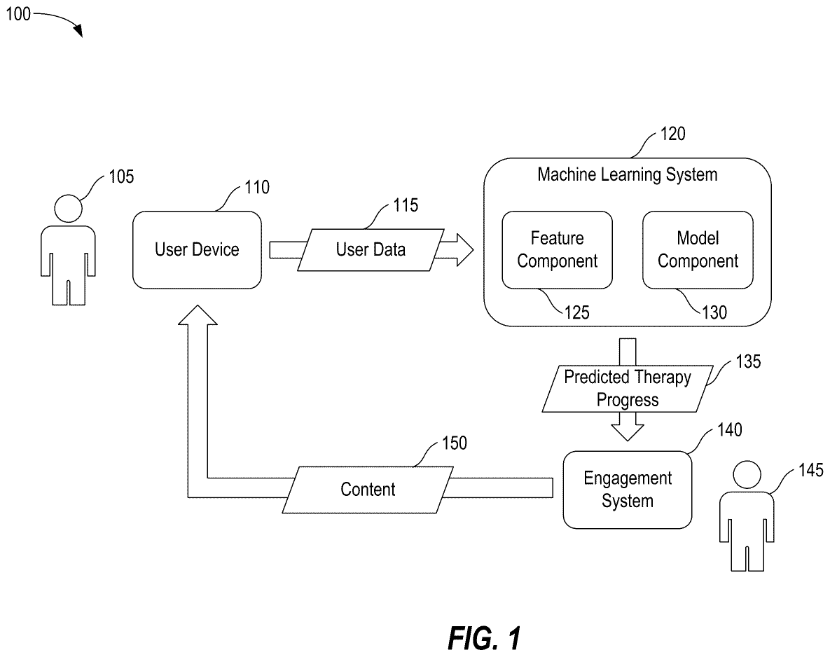

FIG. 1 depicts an example environment 100 for using machine learning to predict and manage therapy progress.

The depicted environment 100 includes a user device 110, machine learning system 120, and engagement system 140. Although depicted as discrete components for conceptual clarity, in some aspects, the depicted components (and others not illustrated) may be combined or distributed across any number of systems. For example, the operations of the machine learning system 120 and engagement system 140 may be combined and performed by a single device or system.

In the illustrated example, the user device 110, machine learning system 120, and engagement system 140 are connected via one or more communication links. For example, the user device 110, machine learning system 120, and engagement system 140 may use one or more networks (which may include wired networks and/or wireless networks), such as the Internet, to intercommunicate. Although the illustrated example depicts directional communication for conceptual clarity (e.g., where the user device 110 transmits data to the machine learning system 120, which transmits data to the engagement system 140, which in turn transmits data to the user device 110), in embodiments, some or all of the communication links may be bidirectional (e.g., the machine learning system 120 may transmit data directly to the user device 110, the user device 110 may transmit data to the engagement system 140, and the like).

In the illustrated example, the user device 110 is associated with or corresponds to a user 105. For example, the user device 110 may be a computing device (e.g., a laptop, a smartphone, a desktop computer, and the like) used by the user 105. The user 105 is an individual associated with a therapy plan. As used herein, a user may be referred to as “associated with” a therapy plan to indicate that they are currently engaged in one, preparing to engage in one, contemplating or considering engaging in one, and the like. In some aspects, the user 105 may be referred to as a “patient” or a “prospective patient”. For example, the user 105 may be a candidate for PAP therapy (e.g., because they believe they may have sleep apnea and would like to take a survey or other test to determine whether a PAP device would help them).

As used herein, a “therapy plan” may include an ongoing therapy (e.g., using a PAP machine during sleep), as well as the enrollment process for such a therapy (e.g., including submitting a survey, taking one or more sleep tests, securing a diagnosis, ordering a PAP machine, and the like). Generally, a therapy plan is defined as a collection, set, or sequence of events or actions that occur or are performed by the user engaged in the plan or by other users. For example, a therapy plan corresponding to enrollment in a PAP therapy may include a stage corresponding to initial interest/investigation (e.g., where the user asks a healthcare professional about their sleep, or takes a survey), a stage where the user participates in a sleep study, and the like.

In the illustrated example, the user device 110 provides a set of user data 115 to the machine learning system 120. The user data 110 can generally include a wide variety of data relating to the user 105, such as their demographics (e.g., age, weight, height, and the like), survey data (e.g., their responses to survey questions), sleep and/or medical history, and the like. In at least one embodiment, the user data 115 is provided to the machine learning system 120 at the start or beginning of the enrollment process for a therapy (e.g., for PAP therapy). For example, the user data 115 may be collected as part of a survey or questionnaire, where the user 105 is a prospective or potential PAP patient and provides the survey responses for purposes of determining whether to proceed with PAP (e.g., to determine whether they have sleep apnea, whether PAP therapy may help, and the like).

As illustrated, the machine learning system 120 accesses, receives, or retrieves the user data 115 and evaluates it to generate a predicted therapy progress 135 for the user 105. As discussed in more detail below, the predicted therapy progress 135 (also referred to as a progress measure in some aspects) generally indicates the expected or predicted progression of the user 105 with respect to the therapy plan. For example, the predicted therapy progress 135 may indicate the probability that the user 105 will complete the plan (or reach a defined minimum stage, such as completing a sleep study), the predicted amount of time that will pass or elapse before the user 105 completes the plan or reaches a defined stage, the predicted amount of resources that will be used to help the user 105 along the plan, and the like.

In an embodiment, the machine learning system 120 uses machine learning to generate the predicted therapy progress 135. In the illustrated example, the machine learning system 120 includes a feature component 125 and a model component 130, which may be implemented using hardware, software, or a combination of hardware and software. Although depicted as discrete components for conceptual clarity, in some aspects, the operations of the feature component 125 and model component 130 may be combined or distributed across any number of components and systems.

In one embodiment, as discussed below in more detail, the feature component 125 performs feature extraction on the user data 110 during runtime and/or training of the model. That is, the feature component 125 may use one or more transformations, preprocessing operations, aggregation operations, or other functions or techniques to identify, generate, and/or extract features or attributes from the user data 115. In some embodiments, the feature component 125 performs this feature extraction based on transformation metadata corresponding to the training of the model. For example, when training the machine learning model, metadata defining or specifying the transformations and preprocessing operations may be generated. In this way, when processing data during runtime, the feature component 125 can replicate the same preprocessing or transformations.

In an embodiment, as discussed below in more detail, the model component 130 may perform model training and/or model inferencing during runtime. For example, the model component 130 may receive or access the relevant features or attributes (e.g., extracted from the user data 115 by the feature component 125), and process them using a machine learning model to generate a predicted therapy progress 135. In some embodiments, during a training stage, the model component 130 can compare the generated prediction against a ground-truth label (e.g., data indicating what stage of the therapy plan was reached or completed by the user), and refine the parameters of the model component 130 based on the difference between the prediction and ground-truth.

In some embodiments, the machine learning system 120 can be used to both train the model, as well as use it during runtime. That is, the same system may perform training and runtime inferencing. In other embodiments, the models may be trained on a first computing system, and then deployed to one or more other systems to perform runtime inferencing.

In the illustrated example, during runtime, the machine learning system 120 outputs the predicted therapy progress 135 to an engagement system 140. In some embodiments, the engagement system 140 is used by subject matter experts (e.g., healthcare professionals, sleep coaches, and the like) to engage with users 105 in order to help them along the therapy plan. For example, in the illustrated example, a user 145 (e.g., a sleep coach) may use the engagement system 140 to review or evaluate the predicted therapy progress 135 of one or more users 105.

In some embodiments, the machine learning system 120 provides the predicted therapy progress 135 of any users 105 that engage in or begin the therapy plan, or otherwise provide user data 115. That is, the machine learning system 120 may generate and provide a respective predicted therapy progress 135 for each user 105, and the user 145 and/or engagement system 140 may determine how to proceed by evaluating these measures. In at least one embodiment, the machine learning system 120 and/or engagement system 140 can transmit, receive, and/or output only a subset of the predicted therapy progresses 135. For example, the machine learning system 120 and/or engagement system 140 may evaluate generated predicted therapy progresses 135 to determine whether they satisfy defined criteria (e.g., a minimum preferred score or probability that the user will complete the therapy plan). In one such embodiment, the user 145 can use the engagement system 140 to identify or review users 105 that have a sufficiently high probability of completing the plan (e.g., of actually beginning PAP therapy).

In the illustrated example, based on the predicted therapy progress 135, the engagement system 140 can selectively or dynamically provide content 150 to the user 105 (via the user device 110). The content 150 generally corresponds to any information, content, prompts, or other data relating to the therapy plan. For example, the content 150 may include an invitation or prompt to begin the next phase of the plan (e.g., to schedule a sleep study), articles giving information about the plan (e.g., discussing PAP therapy and sleep apnea), invitations to discuss the plan with the user 145, and the like.

In some embodiments, the engagement system 140 automatically transmits content 150 to users 105 having a sufficiently high predicted therapy progress 135. In some embodiments, the user(s) 145 can manually provide the content 150 (e.g., based on reviewing the predicted therapy progresses 135). In at least one embodiment, the provided content 150 may vary depending on the particular user 105 and/or predicted therapy progress 135. For example, if the predicted therapy progress 135 is above a first defined threshold, the content 150 may include an invitation to schedule or begin the next phase of the plan. If the predicted therapy progress 135 is below the first threshold but above a second, the content 150 may include informational data (e.g., to inform the user 105 or attempt to persuade them to proceed with the plan).

In some embodiments, the illustrated workflow is performed once for each user 105. For example, at the start of the plan (e.g., after filling out a questionnaire), the machine learning system 120 may generate a predicted therapy progress 135 for the user 105, and the user 145 and/or engagement system 140 may proceed to provide content 150. In some embodiments, the workflow may be repeated for a given user 105. For example. in one embodiment, the machine learning system 120 may iteratively generate new predicted therapy progresses 135 for the user 105, based on new or updated user data 115, for as long as they remain on the therapy plan. For example, the machine learning system 120 may generate a new predicted therapy progress 135 daily, weekly, whenever the user 105 completes a stage of the plan, and the like. In this way, users (e.g., users 145) can continuously monitor the progress of the user 105, improving the probability that they remain on the plan and receive any assistance or content 150 that may help them on their path.

### Example Workflow for Training Machine Learning Models to Predict Therapy Progress

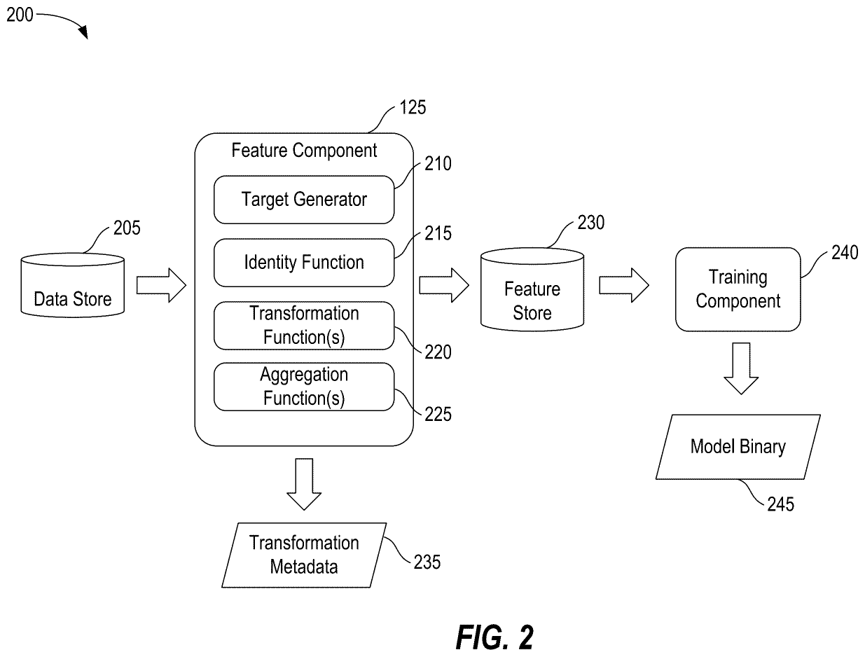

FIG. 2 depicts an example workflow 200 for training machine learning models to predict therapy progress. In some embodiments, the workflow 200 is performed by a machine learning system, such as the machine learning system 120 of FIG. 1.

In the illustrated workflow 200, a feature component 125 (e.g., the feature component 125 of the machine learning system 120 discussed above with reference to FIG. 1) accesses a data store 205 as input. In an embodiment, the data store 205 corresponds to a repository of training data for the machine learning model(s). Although depicted as a single discrete repository for conceptual clarity, in some aspects, the data store 205 may correspond to a collection of various data sources.

In an embodiment, the data store 205 generally includes historical data for one or more users associated with therapy plans. For example, the data store 205 may store, for one or more users who began and/or completed an enrollment process for PAP therapy, corresponding user data, such as their demographics, survey responses, symptoms, and the like. In some embodiments, the data store 205 further includes, for each user reflected in the data, an indication of the therapy progress they made before either completing the plan or terminating participation.

For example, in the case of a PAP enrollment process, the data store 205 may indicate which stage the user reached (e.g., whether they submitted the survey responses, whether they scheduled a sleep study, whether they completed the sleep study, whether they ordered PAP equipment, whether they began using the equipment, and so on). In some embodiments, the data store 205 can additionally or alternatively include any other therapy progress data, such as how much time elapsed before completing (or terminating) the plan or sequence, any indicated reason(s) for terminating prior to completion, and the like.

In some embodiments, the data in the data store 205 may be referred to as exemplars, training exemplars, samples, training samples, and the like, where each exemplar/sample corresponds to the data for a single user with respect to a single attempt to complete the therapy plan. For example, a first exemplar may include the demographics data for a first user at a first point in time when they responded to a survey to seek information about PAP therapy, along with an indication as to how far along the process the first user reached (e.g., the stage or phase that the user achieved or completed). The data store 205 may further include a second exemplar having demographics of a second user who began the process, a third exemplar for a second time when the first user attempted to undergo the process a second time, and so on.

As illustrated, the feature component 125 includes a set of components that perform various data transformations, extractions, aggregations, and the like. Specifically, in the illustrated example, the feature component 125 includes a target generator 210, an identity function 215, one or more transformation functions 220, and one or more aggregation functions 225. Though depicted as discrete components for conceptual clarity, in embodiments, the operations of the target generator 210, identity function 215, transformation functions 220, and aggregation functions 225 may be combined or distributed across any number of systems and components, and may be implemented using hardware, software, or a combination of hardware and software. Additionally, in some aspects, the feature component 125 may or may not include one or more of the depicted components depending on the particular implementation (e.g., the system may not use any aggregation functions 225 for some or all model versions).

In the illustrated workflow 200, the target generator 210 is used to extract or generate a label (also referred to as a target) from the input data in the data store 205. That is, for each exemplar or sample in the data store 205, the target generator 210 can evaluate the data to generate, identify, or extract a target label for the sample. In some embodiments, the target label is determined based at least in part on the desired output of the model. For example, the target generator 210 may determine whether the user completed the entire plan (e.g., completed all stages or phases), specific the stage(s) or phase(s) that the user completed in the therapy plan, the stage or phase where they terminated participation (e.g., the last-completed or achieved stage), and the like. In some embodiments, the target generator 210 may additionally or alternative determine how much time elapsed until the user terminated participation or completed the plan, and the like.

In some embodiments, the target generator 210 generates the label based on a defined minimum amount of progress. For example, a user or administrator may specify that reaching a specified stage in the enrollment process (e.g., completing a sleep study) is a positive result, while failing to reach and/or complete this stage is a negative result. That is, the target generator 210 may generate a binary label (e.g., positive or negative) based on whether the user reached the specified minimum stage of progress. In some embodiments, the target generator 210 may additionally or alternatively generate continuous labels, such as indicating the number of days that elapsed before the user completed the plan (e.g., completed the final stage or phase). As discussed below in more detail, these label(s) can be used as the target variable when training and refining the machine learning model(s).

The identity function 215 may generally correspond to a component that does not alter the input data in the data samples, but simply maps or copies it to be used directly as an input feature. For example, in some embodiments, data such as the user's weight may be directly mapped as an input feature for the model.

The transformation functions 220 can generally correspond to any data transformations applied to input data, including those that involve simple or complex logic or other preprocessing. For example, input data in the data store 205 such as the user's year of birth and the date when the user submitted the survey responses can be used to compute or generate a feature corresponding to the user's age at the time when they began the process. As another example, the transformation functions 220 may include a process to split a multi-selection input data structure (e.g., where the user can enter a list of symptoms) into discrete output features. Generally, the transformation functions 220 can include any transformations or logic, and may map one or more input data elements to one or more output features, to be used as input by the model. The aggregation functions 225 generally correspond to any data aggregation performed on the input data in the data store 205.

In the illustrated example, the feature component 125 outputs a set of labeled features (depicted as residing in a feature store 230) for each exemplar in the data store 205. Although depicted as residing in a discrete repository for conceptual clarity, in some aspects, the features may be stored or maintained in any suitable location and manner. Generally, as discussed above, each record in the feature store 230 corresponds to an input data sample or exemplar, and includes a set of features (generated or extracted by the feature component 125) and a corresponding label (e.g., indicating the user's historical therapy progress).

As illustrated, the feature component 125 can also output, generate, or otherwise provide a set of transformation metadata 235. In an embodiment, the transformation metadata 235 may specify or define the specific transformations or other preprocessing operations that were used to generate the data in the feature store 230. For example, the transformation metadata 235 may indicate the specific techniques or algorithms used, by the target generator 210, to generate the output labels, the specific input elements that are mapped directly by the identity function 215, the specific transformations and aggregations applied by the transformation function(s) 220 and aggregation function(s) 225, and the like.

In the illustrated workflow 200, the transformation metadata 235 may be labeled in such a way that it can be linked to the trained model (e.g., using version numbers). In this way, when the model is stored or deployed for runtime use, the system can also identify and access the relevant transformation metadata 235 defining how the input data should be preprocessed. This ensures that the feature component 125 can use different transformations and preprocessing for different model versions, enabling enhanced flexibility and accuracy.

For example, if multiple machine learning models are trained (e.g., where each region or country has a corresponding machine learning model), the feature component 125 can generate corresponding transformation metadata 235 for each such model. This ensures that, regardless of the specific model version used to process a given set of input data, the appropriate or correct transformations or other preprocessing operations are applied.

In the illustrated example, a training component 240 accesses the feature store 230 to train a machine learning model. In one embodiment, the training component 240 corresponds to or is included as a subcomponent of the model component 130 of FIG. 1. Generally, to train the model, the training component 240 refines one or more parameters of the model (e.g., weights in a neural network) to better fit the output labels generated by the target generator 210. For example, in the case of a neural network architecture, the training component 240 may process the extracted features for a given sample as input in order to generate an output of the model (e.g., a predicted therapy progress measure). By comparing this prediction against the generated ground-truth label, the training component 240 can generate a loss that can be used to refine the model (e.g., using backpropagation and gradient descent).

In embodiments, the training component 240 may train the model until a variety of termination criteria are met, including a maximum time or computational resources spent training, a number of training samples, a determination that no further samples remain, and the like. Once the model is trained, the training component 240 outputs a model binary 245. The model binary 245 is generally a data structure containing the relevant details of the model (e.g., its parameters, hyperparameters, architecture, and the like). That is, the model binary 245 can generally correspond to any data structure containing the relevant information that is used to instantiate or deploy the model for runtime inferencing.

In some embodiments, though not depicted explicitly in the illustrated workflow 200, the model binary 245 and transformation metadata 235 can then be stored (e.g., in a model repository) and/or provided to one or more other systems to be deployed. For example, the machine learning system may provide a link or pointer to, or otherwise enable access to, the model binary 245 for downstream systems to access and use to instantiate a model, which can then be used to provide runtime predictions (e.g., to predict user therapy progression).

In some embodiments, as discussed above, the model binary 245 and transformation metadata 235 are linked, connected, or otherwise associated together. For example, they may be stored a shared location or folder in a model repository, linked, labeled, or named with a shared version number, and the like. In this way, when the model binary 245 is used to instantiate a machine learning model, the corresponding transformation metadata 235 can be accessed and used to deploy the feature extraction operations.

### Example Workflow for Using Machine Learning Models to Predict Therapy Progress

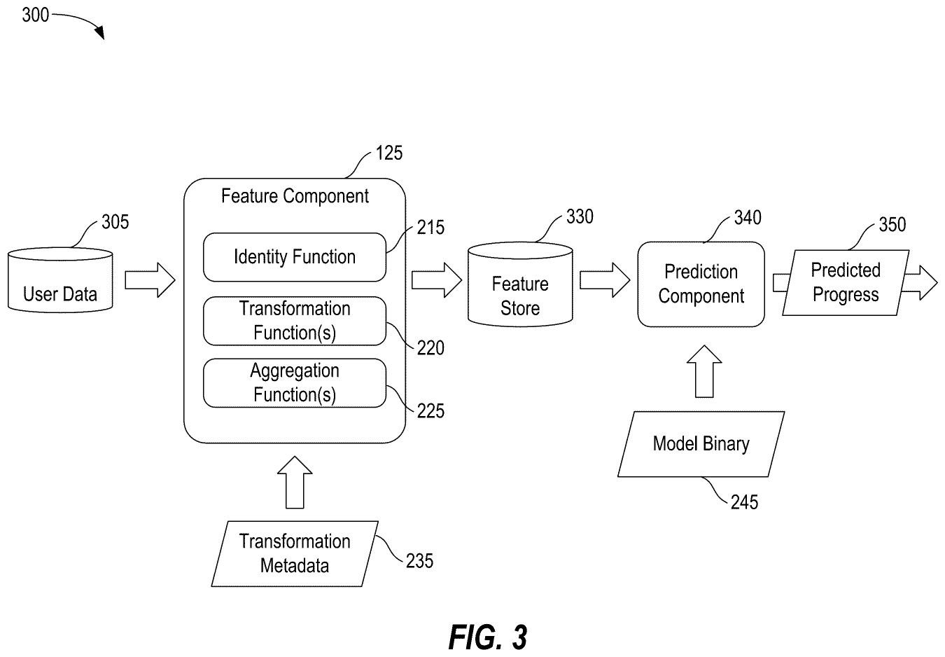

FIG. 3 depicts an example workflow for using machine learning models to predict therapy progress. In some embodiments, the workflow 300 is performed by a machine learning system, such as the machine learning system 120 of FIG. 1. In some embodiments, the workflow 300 is performed after the workflow 200 is performed (e.g., by the machine learning system or by another system) to train the model.

In the illustrated workflow 300, a feature component 125 (e.g., the feature component 125 of the machine learning system 120 discussed above with reference to FIG. 1) accesses user data 305 as input. In some embodiments, the user data 305 corresponds to user data 115 of FIG. 1. Although depicted as a single discrete repository for conceptual clarity, in some aspects, the user data 305 may correspond to a collection of various data sources.

In an embodiment, the user data 305 generally includes data for one or more users associated with therapy plans. For example, the user data 305 may include, for one or more users who have begun or are considering beginning an enrollment process for PAP therapy, corresponding user data, such as their demographics, survey responses, symptoms, and the like.

In some embodiments, the data in the user data 305 may be referred to as input exemplars, data exemplars, input samples, data samples, and the like, where each exemplar/sample corresponds to the data for a single user with respect to a single attempt to complete the therapy plan. For example, a first exemplar may include the demographics data for a first user at a first point in time when they responded to a survey to seek information about PAP therapy, a second exemplar having demographics of a second user who began the process, and the like.

As illustrated, the feature component 125 includes a set of components that perform various data transformations, extractions, aggregations, and the like. Specifically, as discussed above, the feature component 125 includes the identity function 215, the one or more transformation functions 220, and the one or more aggregation functions 225. Though depicted as discrete components for conceptual clarity, in embodiments, the operations of the identity function 215, transformation functions 220, and aggregation functions 225 may be combined or distributed across any number of systems and components, and may be implemented using hardware, software, or a combination of hardware and software. Additionally, in some aspects, the feature component 125 may or may not include one or more of the depicted components depending on the particular implementation (e.g., the system may not use any aggregation functions 225 for some or all model versions).

The operations of the identity function 215, transformation function(s) 220, and aggregation function(s) 225 are discussed in more detail above with reference to FIG. 2. In the illustrated example, the feature component 125 also receives or accesses transformation metadata 235. For example, as discussed above, the transformation metadata 235 may specify or define the specific transformations or other preprocessing operations that were used to generate the data used to train the machine learning model. For example, the transformation metadata 235 may indicate the specific techniques or algorithms used, the specific input elements that are mapped directly by the identity function 215, the specific transformations and aggregations applied by the transformation function(s) 220 and aggregation function(s) 225, and the like.

In some embodiments, as discussed above, the transformation metadata 235 is stored with or alongside the data structure containing the model information (e.g., the parameters and hyperparameters), such that when the model is instantiated, the relevant preprocessing operations can be identified for use.

In the illustrated example, the feature component 125 outputs a set of features (depicted as residing in a feature store 330) for each sample in the user data 305. Although depicted as residing in a discrete repository for conceptual clarity, in some aspects, the features may be stored or maintained in any suitable location and manner, and may be passed directly to the prediction component 340. Generally, as discussed above, each record in the feature store 330 corresponds to an input data sample, and includes a set of features (generated or extracted by the feature component 125).

In the illustrated example, a prediction component 340 accesses the feature store 330 (or receives the features directly) to be processed using a trained machine learning model. In one embodiment, the prediction component 340 corresponds to or is included as a subcomponent of the model component 130 of FIG. 1. In the illustrated example, the prediction component 340 deploy, instantiate, or otherwise access the machine learning model based on the stored model binary 245. As discussed above, the model binary 245 is generally a data structure containing the relevant details of the model (e.g., its parameters, hyperparameters, architecture, and the like). That is, the model binary 245 can generally correspond to any data structure containing the relevant information that is used to instantiate or deploy the model for runtime inferencing.

In one embodiment, the prediction component 340 can access or select the model binary 245 based on a variety of criteria. For example, the prediction component 340 may be configured to select the most-recently trained version of the model. In some embodiments, if different models are used for different locales (e.g., where user data from a first country is used to train a first model, while user data from a second country is used to train a second model), the prediction component 340 can access or identify the model binary 245 corresponding to the model that was trained for the specific country where the user resides.

As illustrated, the prediction component 340 processes the extracted features (which may reside in the feature store 330) for each data sample using a trained machine learning model to generate a corresponding predicted progress 350. For example, as discussed above, the predicted progress may indicate the predicted stage or phase of a sequence of events or actions that the user is predicted to reach or complete, the probability that the user completes a given stage or phase, the predicted amount of time or other resources that will be used to reach the stage and/or complete the sequence, and the like.

As discussed above, the predicted progress 350 may be used for a variety of practical applications, such as to target dynamic interventions (e.g., providing specific content to users who may be unlikely to complete the therapy plan), to reduce resource waste (e.g., to avoid transmitting or sending any further content to a user that is likely to terminate participation, which thereby reduces network congestion and other computational resources), and the like.

### Example Method for Training Machine Learning Models to Predict Therapy Progress

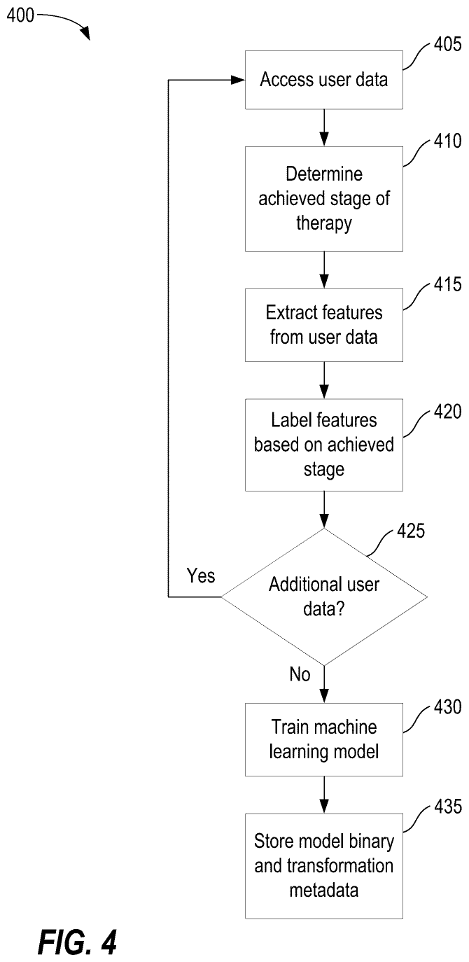

FIG. 4 is a flow diagram depicting an example method 400 for training machine learning models to predict therapy progress. In some embodiments, the method 400 is performed by a machine learning system, such as the machine learning system 120 of FIG. 1. In some embodiments, the method 400 provides additional detail for the workflow 200 of FIG. 2.

At block 405, the machine learning system accesses user data (e.g., via the data store 205 of FIG. 2). For example, as discussed above, the machine learning system may receive, retrieve, or otherwise access historical user data associated with a prior or historical therapy plan. As one example, the machine learning system may access a training exemplar related to a user's engagement or progress in a PAP therapy enrollment sequence. As discussed above, the user data generally includes one or more characteristics or attributes of the user (e.g., demographics data, survey responses, symptoms reported, medical history, and the like), as well as an indication as to the user's progress on the therapy plan (e.g., how far into the plan they progressed, such as identifying the stage or phase where they stopped participating, how long it took them to achieve that stage, and the like).

In embodiments, the machine learning system can select or access the user data using any suitable technique, including randomly or pseudo-randomly. For example, the machine learning system may select or access each exemplar in turn, such that the processing order is irrelevant. Although the illustrated example depicts a sequential and iterative process for conceptual clarity (e.g., accessing and processing each exemplar of user data in sequence), the machine learning system may process some or all of the exemplars in parallel.

At block 410, the machine learning system determines the achieved stage of therapy for the user data. That is, if the therapy plan comprises a set or sequence of stages or phases (e.g., each corresponding to an action or event in the plan), the machine learning system can determine or identify which stage (or set of stages) the user completed successfully. In some embodiments, determining the achieved stage comprises identifying the last or final stage that the user completed. That is, if the therapy includes a sequence of stages, the machine learning system can determine how far into the sequence the user progressed. For example, the machine learning system may determine whether the user completed the final stage, whether the user failed to complete the final stage but completed the penultimate stage, and so on.

At block 415, the machine learning system extracts one or more features from the user data. For example, as discussed above with reference to FIG. 2, the machine learning system may use a defined set of transformations or other preprocessing operations to extract, generate, and/or aggregate features from the user data. In some embodiments, the machine learning system can perform the feature extraction based on a user-defined set of operations (e.g., specified in transformation metadata). As discussed above, this metadata may be maintained or stored along with the trained model in order to ensure that downstream inferencing performs identical preprocessing/feature extraction.

At block 420, the machine learning system labels the extracted/generated features based on the achieved stage of the therapy. In one embodiment, this includes labeling the features to indicate the achieved stage (e.g., to indicate which stage the user reached). In some embodiments, the machine learning system can determine whether the achieved stage satisfies defined minimum criteria (e.g., a minimum enrollment phase, indicating that the exemplar is positive only if the user reached or past the minimum phase). This can allow the machine learning system to label the features using a binary target (e.g., where the label is positive if the user reached and/or completed the minimum stage, and negative if the user did not reach and/or complete the minimum stage).

Although the illustrated example depicts labeling the features based on the achieved stage, in some aspects, the machine learning system can additionally or alternatively label the features based on other data, as discussed above, such as the time that elapsed from when the user began the therapy plan to when they completed it or otherwise terminated participation, the amount and/or types of resources that were used to assist the user along the therapy plan, and the like.

At block 425, the machine learning system determines whether there is at least one additional exemplar of user data remaining to be processed. If so, the method 400 returns to block 405 to access the next exemplar. If not, the method 400 continues to block 430, where the machine learning system trains or refines a machine learning model based on the labeled features. For example, if the model architecture is a neural network, the machine learning system may process the features using the model to generate a predicted output, and compare this output against the generated label to generate a loss, which can then be used to refine the model. In embodiments, a variety of techniques may be used to refine the model parameters, such as stochastic gradient descent, batch gradient descent, and the like.

At block 435, the machine learning system then stores the model binary (e.g., a data structure comprising the relevant model parameters, hyperparameters, and the like) and transformation metadata for subsequent use by inferencing systems (which may include the machine learning system and/or other systems). For example, as discussed above, the machine learning system may store the model and transformation data in a model repository, and/or may transmit it to one or more other systems for instantiation.

Although the illustrated example depicts training a single model, in some aspects, as discussed above, the machine learning system can train multiple models. For example, in at least one embodiment, the machine learning system can train a unique model for each locale or region (e.g., one for each country served by the machine learning system), a unique model for each cultural or other context, and the like. In one such embodiment, at block 405, the machine learning system can access a training exemplar that corresponds to the specific location or culture. By training each model using only data from the corresponding location, the machine learning system can train unique models for each.

### Example Method for Using Machine Learning Models to Predict Therapy Progress

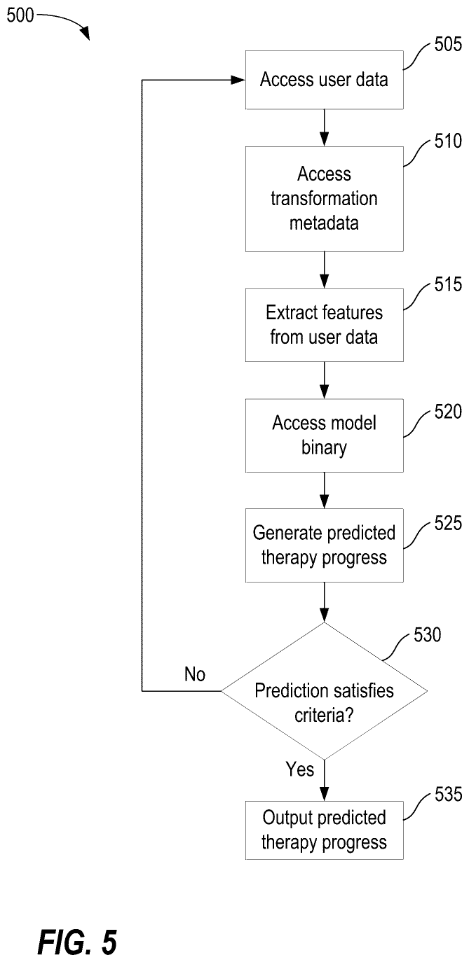

FIG. 5 is a flow diagram depicting an example method 500 for using machine learning models to predict therapy progress. In some embodiments, the method 500 is performed by a machine learning system, such as the machine learning system 120 of FIG. 1. In some embodiments, the method 500 provides additional detail for the workflow 300 of FIG. 3.

At block 505, the machine learning system accesses user data (e.g., the user data 305 of FIG. 3). For example, as discussed above, the machine learning system may receive, retrieve, or otherwise access user data associated with a therapy plan. As one example, the machine learning system may access a data sample related to a user's engagement or beginning of a PAP therapy enrollment sequence. As discussed above, the user data generally includes one or more characteristics or attributes of the user (e.g., demographics data, survey responses, symptoms reported, medical history, and the like). For example, the user data may be collected and/or accessed when the user initially expresses interest in the therapy plan, when a healthcare professional recommends that the user consider the plan, when the user responds to a questionnaire or survey about the plan, and the like.

At block 510, accesses transformation metadata based on the model that will be used to process the user data. For example, the machine learning system may identify which model will be used (e.g., which version) based on a variety of factors, such as identifying the most-recently trained version, identifying the version having a flag or label indicating it is the production or current version, identifying the version trained for the location, region, and/or culture of the specific user represented by the user data, and the like. In an embodiment, at block 510, the machine learning system can identify the transformation metadata that corresponds to this identified model version (e.g., the metadata stored with the model, the metadata having the same version information, and the like).

As discussed above, the transformation metadata can generally indicate or specify any preprocessing operations to be applied to the user data, such as transformations, aggregations, and the like.

At block 515, the machine learning system extracts one or more features from the user data. For example, as discussed above with reference to FIG. 3, the machine learning system may use a defined set of transformations or other preprocessing operations (specified in the transformation metadata) to extract, generate, and/or aggregate features from the user data.

At block 520, the machine learning system accesses the model binary for the relevant model version. That is, the machine learning system retrieves, receives, requests, or otherwise accesses the relevant model (e.g., via a data structure containing the hyperparameters, parameters, and the like) for the user data. In some embodiments, accessing the model binary can include instantiating or deploying the model based on the binary. In some embodiments, accessing the model binary can include using a pre-deployed model (e.g., where the model was already created or instantiated based on a stored data structure).

At block 525, the machine learning system generates a predicted therapy progress measure by processing the user data/features (e.g., the features extracted at block 515) using the model. In some embodiments, as discussed above, the predicted therapy progress indicates whether the user is expected to complete the therapy plan, or to reach some other defined minimum phase. For example, the predicted therapy progress may indicate which stage the user is predicted to reach or complete, the probability that the user will meet or pass the minimum phase, and the like.

In some aspects, the predicted therapy progress can additionally or alternatively include other predictions, as discussed above, such as the time that is predicted to elapse from when the user begins the therapy plan to when they are expected to complete it or otherwise terminate participation, the amount and/or types of resources that are predicted to be used to assist the user along the therapy plan, and the like.

At block 530, the machine learning system determines whether the prediction satisfies one or more defined criteria. For example, as discussed above, the machine learning system may determine whether the predicted progress indicates that the user is expected to reach or complete the defined minimum phase, whether the probability of this success is above a defined threshold, and the like. If the machine learning system determines that the criteria are not satisfied (e.g., because the user is predicted to not complete or reach the defined phase), the method 500 returns to block 505 to receive or access user data for another user.

That is, in the illustrated example, the machine learning system can refrain from further processing or evaluation of the user data, based on determining that the prediction does not satisfy the criteria. In this way, the machine learning system can prevent or reduce resource waste (including manual effort, time consumed, computational resources, and the like) for users that are unlikely to advance sufficiently in the therapy plan.

If the machine learning system determines that the prediction satisfies the criteria, the method 500 continues to block 535, where the machine learning system outputs the predicted therapy progress. For example, the machine learning system may provide or display the predicted progress to a user (e.g., a subject matter expert such as a healthcare professional or a sleep coach), allowing the expert to engage with the user in order to facilitate participation or enrollment in the therapy plan.

Although the illustrated example depicts selectively outputting the predicted progress, in some aspects, the machine learning system may refrain from such filtering and may output all of the generated predictions. For example, the machine learning system may provide the predicted progress measure for each of a set of prospective or potential therapy users, allowing the user (e.g., a sleep coach) to review them, filter them, sort them, and the like based on these predictions. This may allow more robust targeting of limited resources.

### Example Method for Training Machine Learning Models

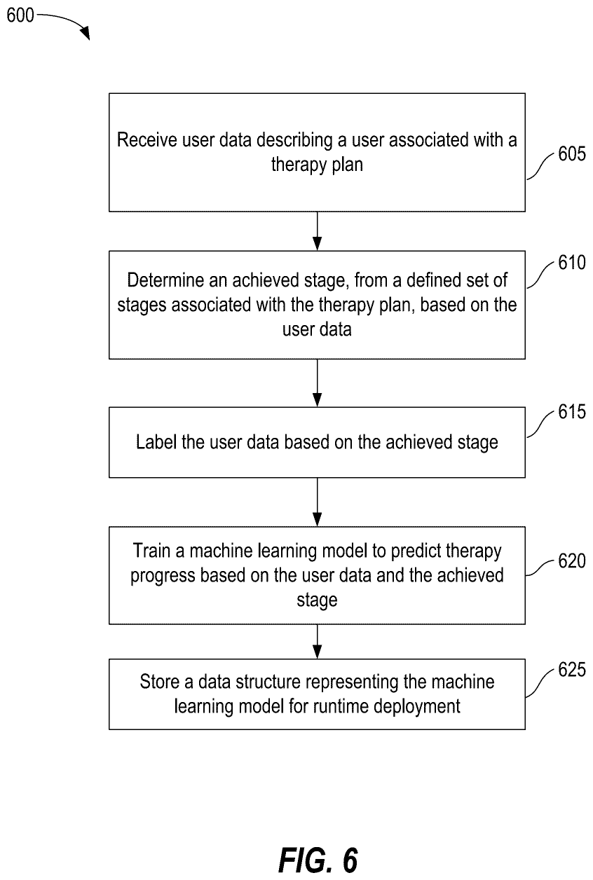

FIG. 6 is a flow diagram depicting an example method 600 for training machine learning models. In some embodiments, the method 600 is performed by a machine learning system, such as the machine learning system 120 of FIG. 1.

At block 605, user data describing a user associated with a therapy plan (e.g., data store 205 of FIG. 2) is received.

At block 610, an achieved stage, from a defined set of stages associated with the therapy plan, is determined (e.g., by a target generator 210 of FIG. 2) based on the user data.

At block 615, the user data is labeled based on the achieved stage.

At block 620, a machine learning model is trained to predict therapy progress based on the user data and the achieved stage.

At block 625, a data structure representing the machine learning model (e.g., the model binary 245 of FIG. 2) is stored for runtime deployment.

### Example Method for Predicting Therapy Progress

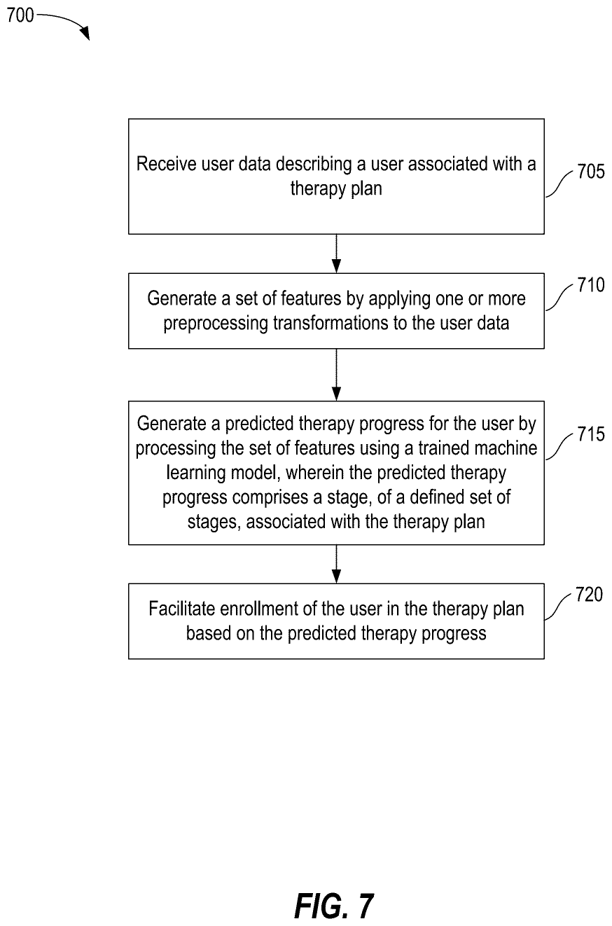

FIG. 7 is a flow diagram depicting an example method 700 for predicting therapy progress. In some embodiments, the method 600 is performed by a machine learning system, such as the machine learning system 120 of FIG. 1.

At block 705, user data describing a user associated with a therapy plan (e.g., user data 115 of FIG. 1) is received.

At block 710, a set of features is generated (e.g., by feature component 125 of FIG. 1) by applying one or more preprocessing transformations to the user data.

At block 715, a predicted therapy progress for the user (e.g., predicted therapy progress 135 of FIG. 1) is generated by processing the set of features using a trained machine learning model, wherein the predicted therapy progress comprises a stage, of a defined set of stages, associated with the therapy plan.

At block 720, enrollment of the user in the therapy plan is facilitated based on the predicted therapy progress (e.g., via content 150 of FIG. 1).

### Example Computing Device for Therapy Progression Prediction

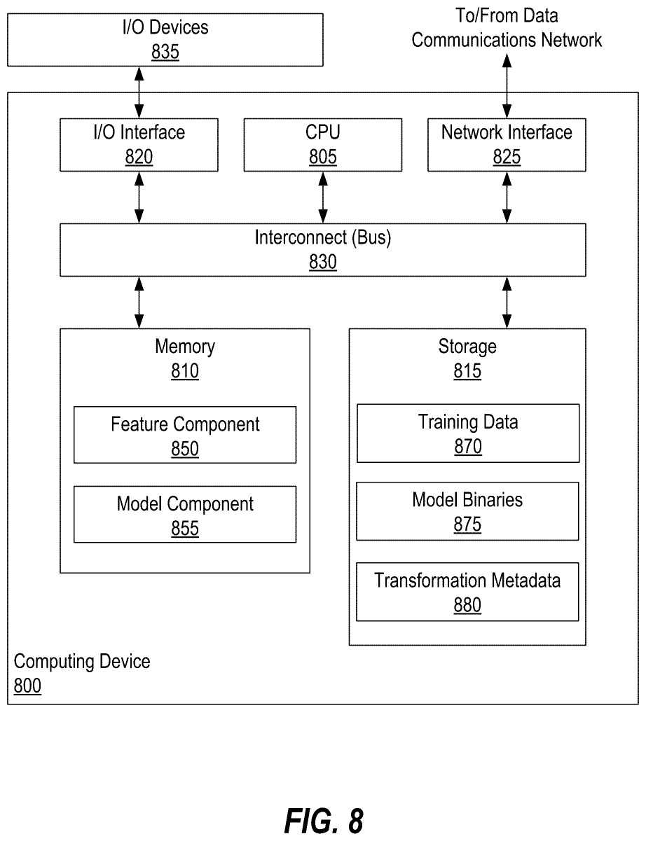

FIG. 8 depicts an example computing device 800 configured to perform various aspects of the present disclosure. Although depicted as a physical device, in embodiments, the computing device 800 may be implemented using virtual device(s), and/or across a number of devices (e.g., in a cloud environment). In one embodiment, the computing device 800 corresponds to one or more systems in a healthcare platform, such as a machine learning system (e.g., machine learning system 120 of FIG. 1).

As illustrated, the computing device 800 includes a CPU 805, memory 810, storage 815, a network interface 825, and one or more I/O interfaces 820. In the illustrated embodiment, the CPU 805 retrieves and executes programming instructions stored in memory 810, as well as stores and retrieves application data residing in storage 815. The CPU 805 is generally representative of a single CPU and/or GPU, multiple CPUs and/or GPUs, a single CPU and/or GPU having multiple processing cores, and the like. The memory 810 is generally included to be representative of a random access memory. Storage 815 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 835 (such as keyboards, monitors, etc.) are connected via the I/O interface(s) 820. Further, via the network interface 825, the computing device 800 can be communicatively coupled with one or more other devices and components (e.g., via a network, which may include the Internet, local network(s), and the like). As illustrated, the CPU 805, memory 810, storage 815, network interface(s) 825, and I/O interface(s) 820 are communicatively coupled by one or more buses 830.

In the illustrated embodiment, the memory 810 includes a feature component 850 and a model component 855, which may perform one or more embodiments discussed above. Although depicted as discrete components for conceptual clarity, in embodiments, the operations of the depicted components (and others not illustrated) may be combined or distributed across any number of components. Further, although depicted as software residing in memory 810, in embodiments, the operations of the depicted components (and others not illustrated) may be implemented using hardware, software, or a combination of hardware and software.

In one embodiment, the feature component 850 may be used to extract and/or generate features based on user data, as discussed above. For example, the feature component 850 (which may correspond to the feature component 125 of FIGS. 1-3) may use and/or define transformation data specifying which preprocessing operations to apply in order to generate the relevant features for the user data. In one embodiment, the model component 855 may be used to train, refine, and/or use trained machine learning models, as discussed above. For example, the model component 855 (which may correspond to the model component 130 of FIG. 1, the training component 240 of FIG. 2, and/or the prediction component 340 of FIG. 3) may train machine learning models to predict therapy progress, and/or use trained models to process user data to predict therapy progress.

In the illustrated example, the storage 815 includes training data 870, one or more model binaries 875, and one or more corresponding sets of transformation metadata 880. In one embodiment, the training data 870 (which may correspond to data store 205 of FIG. 2) may include any data used to train, refine, or test machine learning models, as discussed above. The model binaries 875 may generally include any data structure representing a machine learning model (e.g., model definitions, parameters, hyperparameters, and the like), such as stored in a model registry, as discussed above. The transformation metadata 880 generally corresponds to the preprocessing steps used in conjunction with trained models, such as how data elements in the user data should be transformed, extracted, aggregated, or otherwise prepared for processing using the model, as discussed above. Although depicted as residing in storage 815 for conceptual clarity, the training data 870, model binaries 875, and transformation metadata 880 may be stored in any suitable location, including memory 810 or in one or more remote systems distinct from the computing device 800.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., machine learning system 120 of FIG. 1) or related data available in the cloud. For example, the machine learning system could execute on a computing system in the cloud and train, deploy, and/or monitor machine learning models to predict therapy progress. In such a case, the machine learning system could maintain the models and/or transformation metadata in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

### EXAMPLE CLAUSES

Implementation examples are described in the following numbered clauses:

Clause 1: A method, comprising: receiving user data describing a user associated with a therapy plan; determining an achieved stage, from a defined set of stages associated with the therapy plan, based on the user data; labeling the user data based on the achieved stage; training a machine learning model to predict therapy progress based on the user data and the achieved stage; and storing a data structure representing the machine learning model for runtime deployment.

Clause 2: The method of Clause 1, wherein: the therapy plan comprises a positive airway pressure (PAP) therapy, the defined set of stages comprise a sequence of enrollment phases for the PAP therapy, and the achieved stage indicates whether the user has begun the PAP therapy.

Clause 3: The method of any one of Clauses 1-2, wherein labeling the user data based on the achieved stage comprises: identifying a minimum enrollment phase defined as a positive result; and labeling the user data based on whether the achieved stage satisfies the minimum enrollment phase.

Clause 4: The method of any one of Clauses 1-3, wherein the user data comprises one or more of: (i) demographics of the user, (ii) an indication of sleep quality of the user, (iii) an indication of motivation of the user with respect to the therapy plan, or(iv) one or more symptoms experienced by the user.

Clause 5: The method of any one of Clauses 1-4, further comprising: applying one or more preprocessing transformations to the user data, prior to training the machine learning model; and storing transformation metadata defining the one or more preprocessing transformations along with the data structure for runtime deployment.

Clause 6: The method of any one of Clauses 1-5, further comprising deploying the model based on the stored data structure and transformation metadata.

Clause 7: A method, comprising: receiving user data describing a user associated with a therapy plan; generating a set of features by applying one or more preprocessing transformations to the user data; generating a predicted therapy progress for the user by processing the set of features using a trained machine learning model, wherein the predicted therapy progress comprises a stage, of a defined set of stages, associated with the therapy plan; and facilitating enrollment of the user in the therapy plan based on the predicted therapy progress.

Clause 8: The method of Clause 7, further comprising accessing the trained machine learning model based on a stored data structure and transformation metadata defining the one or more preprocessing transformations.

Clause 9: The method of any one of Clauses 7-8, wherein: the therapy plan comprises a positive airway pressure (PAP) therapy, and the predicted therapy progress indicates whether the user is predicted to complete enrollment in the PAP therapy.

Clause 10: The method of any one of Clauses 7-9, wherein: the defined set of stages comprise a sequence of enrollment phases, and the predicted therapy progress indicates whether the user is predicted to reach a minimum enrollment phase defined as a positive result.

Clause 11: The method of any one of Clauses 7-10, wherein the user data comprises one or more of: (i) demographics of the user, (ii) an indication of sleep quality of the user, (iii) an indication of motivation of the user with respect to the therapy plan, or (iv) one or more symptoms experienced by the user.

Clause 12: The method of any one of Clauses 7-11, further comprising: determining whether the user has completed enrollment in the therapy plan; and refining the trained machine learning model based on whether the user has completed enrollment in the therapy plan.

Clause 13: The method of any one of Clauses 7-12, wherein facilitating enrollment of the user in the therapy plan based on the predicted therapy progress comprises, in response to determining that the predicted therapy progress satisfies defined criteria, outputting the predicted therapy progress to a subject matter expert.

Clause 14: A system, comprising: a memory comprising computer-executable instructions; and one or more processors configured to execute the computer-executable instructions and cause the processing system to perform a method in accordance with any one of Clauses 1-13.

Clause 15: A system, comprising means for performing a method in accordance with any one of Clauses 1-13.

Clause 16: A non-transitory computer-readable medium comprising computer-executable instructions that, when executed by one or more processors of a processing system, cause the processing system to perform a method in accordance with any one of Clauses 1-13.

Clause 17: A computer program product embodied on a computer-readable storage medium comprising code for performing a method in accordance with any one of Clauses 1-13.

What is claimed is:

1. A method, comprising:
receiving user data describing a user associated with a therapy plan;
determining an achieved stage, from a defined set of stages associated with the therapy plan, based on the user data;
labeling the user data based on the achieved stage;
training a machine learning model to predict therapy progress based on the user data and the achieved stage; and
storing a data structure representing the machine learning model for runtime deployment.

2. The method of claim 1, wherein:
the therapy plan comprises a positive airway pressure (PAP) therapy,
the defined set of stages comprise a sequence of enrollment phases for the PAP therapy, and
the achieved stage indicates whether the user has begun the PAP therapy.

3. The method of claim 2, wherein labeling the user data based on the achieved stage comprises:
identifying a minimum enrollment phase defined as a positive result; and
labeling the user data based on whether the achieved stage satisfies the minimum enrollment phase.

4. The method of claim 1, wherein the user data comprises one or more of:
(i) demographics of the user,
(ii) an indication of sleep quality of the user,
(iii) an indication of motivation of the user with respect to the therapy plan, or
(iv) one or more symptoms experienced by the user.

5. The method of claim 1, further comprising:
applying one or more preprocessing transformations to the user data, prior to training the machine learning model; and
storing transformation metadata defining the one or more preprocessing transformations along with the data structure for runtime deployment.

6. The method of claim 5, further comprising deploying the model based on the stored data structure and transformation metadata.

7. A method, comprising:
receiving user data describing a user associated with a therapy plan;
generating a set of features by applying one or more preprocessing transformations to the user data;
generating a predicted therapy progress for the user by processing the set of features using a trained machine learning model, wherein the predicted therapy progress comprises a stage, of a defined set of stages, associated with the therapy plan; and
facilitating enrollment of the user in the therapy plan based on the predicted therapy progress.

8. The method of claim 7, further comprising accessing the trained machine learning model based on a stored data structure and transformation metadata defining the one or more preprocessing transformations.

9. The method of claim 7, wherein:
the therapy plan comprises a positive airway pressure (PAP) therapy, and
the predicted therapy progress indicates whether the user is predicted to complete enrollment in the PAP therapy.

10. The method of claim 9, wherein:
the defined set of stages comprise a sequence of enrollment phases, and
the predicted therapy progress indicates whether the user is predicted to reach a minimum enrollment phase defined as a positive result.

11. The method of claim 7, wherein the user data comprises one or more of:
(i) demographics of the user,
(ii) an indication of sleep quality of the user,
(iii) an indication of motivation of the user with respect to the therapy plan, or
(iv) one or more symptoms experienced by the user.

12. The method of claim 7, further comprising:
determining whether the user has completed enrollment in the therapy plan; and
refining the trained machine learning model based on whether the user has completed enrollment in the therapy plan.

13. The method of claim 7, wherein facilitating enrollment of the user in the therapy plan based on the predicted therapy progress comprises, in response to determining that the predicted therapy progress satisfies defined criteria, outputting the predicted therapy progress to a subject matter expert.

14. A non-transitory computer-readable medium comprising computer-executable instructions that, when executed by one or more processors of a processing system, cause the processing system to perform an operation comprising:
receiving user data describing a user associated with a therapy plan;
generating a set of features by applying one or more preprocessing transformations to the user data;
generating a predicted therapy progress for the user by processing the set of features using a trained machine learning model, wherein the predicted therapy progress comprises a stage, of a defined set of stages, associated with the therapy plan; and
facilitating enrollment of the user in the therapy plan based on the predicted therapy progress.

15. The non-transitory computer-readable medium of claim 14, the operation further comprising accessing the trained machine learning model based on a stored data structure and transformation metadata defining the one or more preprocessing transformations.

16. The non-transitory computer-readable medium of claim 14, wherein:
the therapy plan comprises a positive airway pressure (PAP) therapy, and
the predicted therapy progress indicates whether the user is predicted to complete enrollment in the PAP therapy.

17. The non-transitory computer-readable medium of claim 16, wherein:
the defined set of stages comprise a sequence of enrollment phases, and
the predicted therapy progress indicates whether the user is predicted to reach a minimum enrollment phase defined as a positive result.

18. The non-transitory computer-readable medium of claim 14, wherein the user data comprises one or more of:
(i) demographics of the user,
(ii) an indication of sleep quality of the user,
(iii) an indication of motivation of the user with respect to the therapy plan, or
(iv) one or more symptoms experienced by the user.

19. The non-transitory computer-readable medium of claim 14, the operation further comprising:
determining whether the user has completed enrollment in the therapy plan; and
refining the trained machine learning model based on whether the user has completed enrollment in the therapy plan.

20. The non-transitory computer-readable medium of claim 14, wherein facilitating enrollment of the user in the therapy plan based on the predicted therapy progress comprises, in response to determining that the predicted therapy progress satisfies defined criteria, outputting the predicted therapy progress to a subject matter expert.
